(19) United States
(12) Patent Application Publication
(10) Pub. No.: US 2015/0150538 A1
(43) Pub. Date: Jun. 4, 2015

(54) METHOD FOR IMPROVED DETERMINATION OF MATERNAL HEART RATE AND FETAL MONITORING SYSTEM THERETO

(71) Applicant: KONINKLIJKE PHILIPS N.V., EINDHOVEN (NL)

(72) Inventors: Stefan Benjamin Reuter, Sindelfingen (DE); Markus Wohlschlager, Sindelfingen (DE); Hansjoerg Geywitz, Kusterdingen (DE)

(21) Appl. No.: 14/413,424

(22) PCT Filed: Jul. 2, 2013
(86) PCT No.: PCT/IB2013/055420

§ 371 Date: Jan. 8, 2015

Related U.S. Application Data
(60) Provisional application No. 61/670,648, filed on Jul. 12, 2012.

(51) Int. Cl. A61B 8/08; A61B 5/11; A61B 8/02

(52) U.S. Cl. CPC A61B 8/5223; A61B 8/02; A61B 8/0866; A61B 8/488; A61B 5/11

(57) ABSTRACT

A method for determining at least one maternal physiological parameter out of a heart rate and a breathing rate, comprising the following steps: (a) attaching at least two transducers units (16, 18) of a fetal monitoring system (10) to a maternal abdomen (14); (b) evaluating an acceleration signal regarding at least one out of frequency, amplitude, and signal pattern to derive an acceleration signal corresponding to the at least one maternal physiological parameter; a fetal monitoring system (10), comprising at least two transducers units (16, 18), at least one transducer (24, 26, 28) provided for taking up physiological parameters, at least a first signal processing unit (34), and at least one acceleration sensor (30, 32) different from the at least two transducers (24, 26, 28), wherein the at least one acceleration sensor (30, 32), in at least one operating mode, is rigidly mounted inside a housing (20, 22) of one of the at least two transducer units (16, 18); and wherein the first signal processing unit (34) is provided to evaluate the acceleration signal regarding at least one out of frequency, amplitude, and signal pattern to derive an acceleration signal corresponding to at least one maternal physiological parameter; and a transducer unit (16, 18) for use in a fetal monitoring system (10).

### FIELD OF THE INVENTION

The invention pertains to a method for determining at least one maternal physiological parameter out of a heart rate and a breathing rate by using a fetal monitoring system, to a fetal monitoring system, and to a sensor unit for use in a fetal monitoring system.

### BACKGROUND OF THE INVENTION

In the field of fetal monitoring, i.e. the field of measurement and visualization of more than one physiological parameter of unborn human beings, it is known to use monitoring systems comprising multiple transducers for measuring uterine activity and a fetal heart beat. Basically two methods are applied:

An external or indirect method employs a use of external transducers placed on the maternal abdomen. Typically, ultrasound (US) Doppler transducers are used in this category, wherein high frequency sound waves reflect mechanical action of a fetal heart.

The other method is an internal or direct method that uses a spiral electrode to convert a fetal electrocardiogram obtained from a presenting part of the unborn. This method can be used only when the presenting part is accessible and identifiable. Because of the limited scope of application for the invention, this method shall therefore not be considered in further detail herein.

Fetal monitoring systems of the prior art also provide measurements for maternal parameters like electro-cardiogram (ECG), oxygen saturation by pulse oximetry (SpO2), blood pressure (NBP) or temperature. Generally speaking, a designer of fetal monitoring systems has to make a trade-off between providing extensive monitoring and facilitating a most natural birth without hindering cables and sensors.

Monitoring systems using ultrasound Doppler technology are normally able to accurately calculate the fetal heart rate. However, a Doppler signal is composed of reflections from all moving structures in a maternal body. Normally, these stem from the fetal heart beat only, but also reflections from maternal vessels like the aorta or other abdominal vessels may contribute to the Doppler signal. In most cases, maternal and fetal heart rates are easy to distinguish, because the frequency of a fetal signal is much higher than a maternal heart rate. In case of maternal stress or drug delivery as well as in the case of decreased fetal heart rates, the two heart rates may converge or even become inverted. Because the fetal monitoring technology cannot detect a difference between a fetal and a maternal signal source when applying a transducer for picking up a fetal heart rate, producing a continuous maternal heart rate trace is the recommended method.

Most fetal monitoring systems have built-in comparison algorithms for identifying duplication of heart rates. This feature helps to detect trace coincidences. Question marks are automatically printed whenever two recorded heart rate traces show similarities over a certain amount of time.

The methods described above for obtaining the maternal heart rate require either additional sensors or at least additional electrodes. Electrodes and sensors add additional cables, thus increasing patient and caregiver inconvenience. As a result, any method that adds additional sensors or electrodes is not well accepted.

It is therefore desirable to provide a method for determining at least one maternal physiological parameter such as a maternal heart rate that is both reliable and robust without employing additional sensors, electrodes, or the like.

### SUMMARY OF THE INVENTION

In one aspect of the present invention, the object is achieved by a method for determining at least one maternal physiological parameter out of a heart rate and a breathing rate, comprising the following steps:
(a) attaching at least two transducers units (16, 18) of a fetal monitoring system to a maternal abdomen, the fetal monitoring system further comprising
at least one transducer provided for taking up physiological parameters and converting them into corresponding signals;
at least one signal processing unit provided to process signals;
at least one acceleration sensor provided for converting mechanical acceleration at the maternal abdomen into a corresponding acceleration signal, wherein the at least one acceleration sensor is different from the at least one transducer, and wherein the at least one acceleration sensor, in at least one operating mode, is rigidly mounted inside a housing that at least partially encompasses at least one of the at least two transducers;
(b) evaluating the acceleration signal regarding at least one out of frequency, amplitude, and signal pattern to derive an acceleration signal corresponding to the at least one maternal physiological parameter.

The phrase “transducer”, as used in this application, shall be understood particularly as a means for converting one form of energy into another form of energy, in particular, for converting mechanical energy or radiant energy into electric energy, and vice versa. Exemplary transducers that shall be encompassed are passively operated transducers such as pressure-sensitive sensors, as well as transducers that are actively operated at one time of operation and are passively operated at another time of operation, such as ultrasound Doppler transducers.

By this method, an additional signal from an independent source can be obtained that allows for confirmation of physiological data and thus, for prevention of mistakes. It therefore provides a reduced risk of misinterpreting the fetal physiological parameter, while maintaining operating comfort to caregivers and ease to the child-bearing or birth giving mother, as the maternal physiological parameter can be determined in a reliable and robust manner without employing additional sensors, electrodes, or the like.

In a further aspect of the invention, the method further comprises the steps of (c) determining and tracking a cross-channel verification between the acceleration signal and at least one signal of the at least one transducer corresponding to a fetal heart rate; and (d) depending on a result of the cross-channel verification, activating an alert signal.

The phrase “cross-channel verification”, as used in this application, shall be understood particularly as any method of obtaining a measure of similarity between two signals in a timely domain, and shall in particular encompass methods derived from a frequency analysis of the two signals. By that, the acceleration signal can be employed as a signal from an independent source to check against a fetal heart rate signal obtained from one of the transducers, and a risk for misinterpreting the maternal physiological parameter, such as a maternal heart rate, as a fetal physiological parameter such as a fetal heart rate, is substantially reduced.

In yet another aspect of the present invention, the method further comprises the step of (c2) selecting one out of the transducer signals and the accelerator signal, based on an evaluation of at least one out of signal history and signal quality.

Thereby, a desired continuous trace of the maternal physiological parameter can be kept to a higher degree, with a lower number of gaps in the trace, and the risk for misinterpreting the maternal physiological parameter as the fetal physiological parameter can be reduced further.

It is another object of the invention to provide a fetal monitoring system, comprising at least two transducer units, at least one transducer provided for taking up physiological parameters and converting them into corresponding signals, at least a first signal processing unit provided to process signals, and at least one acceleration sensor provided for converting mechanical acceleration of a maternal abdomen into a corresponding acceleration signal, wherein the at least one acceleration sensor is different from the at least one transducer, wherein the at least one acceleration sensor, in at least one operating mode, is rigidly mounted inside a housing of one of the at least two transducer units, and wherein the first signal processing unit is provided to evaluate the acceleration signal regarding at least one out of frequency, amplitude, and signal pattern to derive an acceleration signal corresponding to at least one maternal physiological parameter.

An additional signal from an independent source can be obtained from the at least one acceleration sensor that allows for confirmation of physiological data and thus, for prevention of mistakes. The fetal monitoring systems may provide a reduced risk of misinterpreting the maternal physiological parameter, while maintaining operating comfort to caregivers and ease to the child-bearing or birth-giving mother, as the maternal physiological parameter can be determined in a reliable and robust manner without employing additional sensors, electrodes, or the like.

Preferably, the first signal processing unit may reside in the housing of the one of the transducer units the acceleration sensor is rigidly mounted in, and may be designed to receive the acceleration signal as an input signal. Alternatively, a second signal processing unit may be located in the housing of the one of the transducer units the acceleration sensor is rigidly mounted in, that may be assigned exclusively for evaluating the acceleration signal.

A continuous real-time monitoring can be obtained by furnishing the fetal monitoring system with a signal selector unit that is provided for selecting one out of the transducer signals and the accelerator signal, based on an evaluation of at least one out of signal history and signal quality. This may also allow for an unambiguous attribution of the selected signal to the at least one maternal physiological parameter.

In a preferred embodiment, the signal selector unit comprises a software module that is provided for carrying out the steps of determining and tracking the cross-channel verification and the activating of the alert, depending on the result of the cross-channel verification, and/or the step of selecting one out of the transducer signals and the accelerator signal. The steps are converted into a program code that is implementable in and executable by the fetal monitoring system. Thereby, a flexible adjustment, easy portability of the method and method parameters and a uniformity of products may readily be attainable.

The fetal monitoring system may further comprise a cross-channel verification unit provided for determining a cross-channel verification between traces of at least two of the signals in an at least partially continuous time interval. Thereby, a risk of misinterpreting the maternal physiological parameter as a fetal physiological parameter may be highly reducible.

In a preferred embodiment, the at least one acceleration sensor is designed as a micro-electromechanical system (MEMS). Thus, a robust and reliable solution for an acceleration sensor can be provided, which is rigidly mountable inside the housing of one of the at least two transducers in an easy manner.

Preferably, the at least one acceleration sensor is provided for converting mechanical acceleration into corresponding acceleration signals in three substantially orthogonal directions, by which a complete coverage of monitoring physiological parameters for all potential relative arrangements of the transducers of the fetal monitoring system and the subject may be achieved.

It is another object of the invention to provide a transducer unit for use in a fetal monitoring system. The transducer unit comprises at least one transducer provided for taking up physiological parameters and converting them into a corresponding signal, a housing that at least partially encompasses the transducer, and at least one acceleration sensor provided for converting mechanical acceleration into a corresponding acceleration signal, wherein the at least one acceleration sensor, in at least one operating mode, is rigidly mounted inside the housing, so that no additional external cabling is required.

### BRIEF DESCRIPTION OF THE DRAWINGS

These and other aspects of the invention will be apparent from and elucidated with reference to the embodiments described hereinafter. Such embodiment does not necessarily represent the full scope of the invention, however, and reference is made therefore to the claims and herein for interpreting the scope of the invention.

In the drawings:

FIG. 1 shows a schematic plan view of two transducer units of a fetal monitoring system arranged in preparation of a state of operation at a mother prior to birth, and

FIG. 2 illustrates a schematic functional diagram of the fetal monitoring system pursuant to FIG. 1.

### DETAILED DESCRIPTION OF EMBODIMENTS

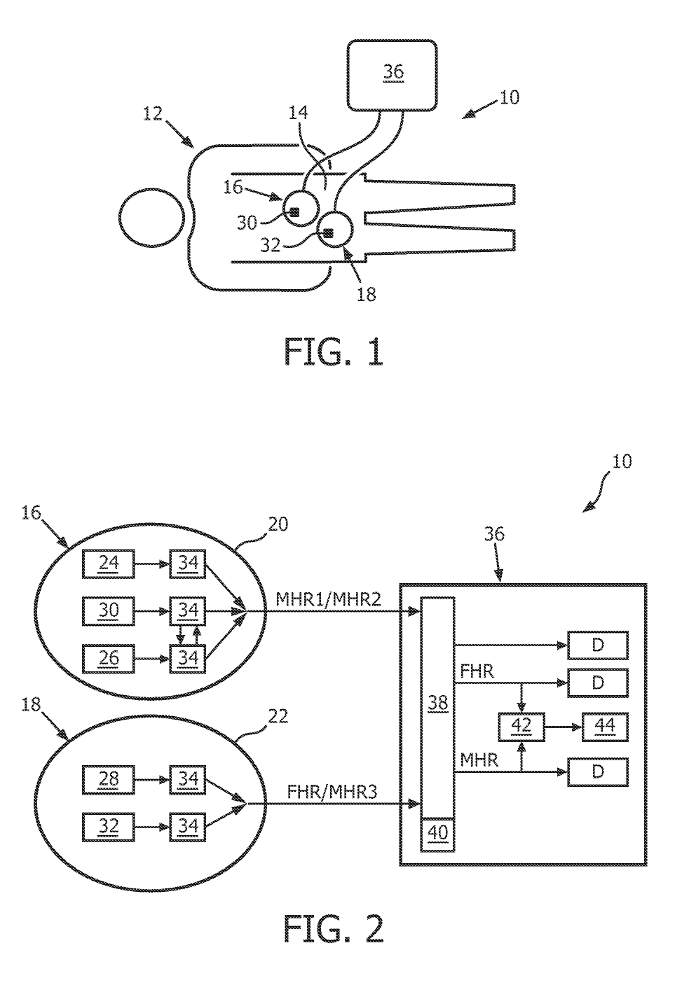

FIG. 1 shows a schematic plan view of a fetal monitoring system 10 comprising two transducer units 16, 18 arranged in preparation of a state of operation at a mother 12 prior to birth. The two transducer units 16, 18 are designed for being attached to the mother's abdomen 14 with a belt (not shown).

The first transducer unit 16 comprises a housing 20 that partially encompasses a transducer 24 designed as a tocodynamometer having a flat area that is in contact with the abdomen 14. The tocodynamometer is provided for taking up a first maternal physiological parameter, namely a uterine pressure, which is converted by the tocodynamometer into a corresponding uterine pressure signal. Further, the first transducer unit 16 comprises another transducer 26 that is designed as an optical sensor which is integrated in the first transducer unit 16 at a lower outer surface of the housing 20. The optical transducer 26 is therefore also in contact with the abdomen 14. It uses an optical method to detect a pulsation of dermal blood vessels to derive a maternal heart rate signal MHR1. The pulsation in the abdominal skin is extremely weak, and the optical transducer 26, although having a number of advantages in other regards, is very susceptive to movements between transducer 26 and skin, which are caused by movements of the mother 12 or even by her coughing or laughing. This might result in gaps in a trace of the maternal heart rate MHR1.

The second transducer unit 18 comprises another housing 22 that partially encompasses a transducer 28 designed as an ultrasonic Doppler sensor, which is provided for taking up another physiological parameter, namely a fetal heart rate FHR. As mentioned in the introduction, a signal from the ultrasonic Doppler sensor that is intended to correspond to the fetal heart rate FHR might be affected by reflections from maternal vessels like the aorta or other abdominal vessels if not correctly adjusted or by a shift of a position of the unborn.

The first transducer unit 16 and the second transducer unit 18 each comprise an acceleration sensor 30, 32 that is different from the transducers 24, 26, 28 of the respective transducer unit 16, 18. In a state of operation, each one of the acceleration sensors 30, 32, which is provided for converting mechanical acceleration of the maternal abdomen 14 into a corresponding acceleration signal, is rigidly mounted inside the housing 20, 22 of the respective transducer unit 16, 18.

The acceleration sensor 32 of the second transducer unit 18 is insensitive to any material between the sensor and skin like water or an acoustic coupling gel, which is required for the ultrasonic Doppler sensor to work properly. Thus, a usage of the ultrasonic Doppler sensor is not restricted by the acceleration sensor 32 in any way.

Each of the acceleration sensors 30, 32 is designed as a micro-electro-mechanical system (MEMS) of a type based on the cantilever beam and proof mass-principle. This type of sensor is well known to the one of skills in the art and shall therefore not be described in further detail herein. The acceleration sensors 30, 32 are provided for converting mechanical acceleration into corresponding acceleration signals in three orthogonal directions, so that movements of the maternal abdomen 14 in any direction can be detected. As the transducer units 16, 18 are located at different positions at the abdomen 14 and with different relative orientation with regard to the abdomen 14, both acceleration sensors 30, 32 will acquire slightly different and independent signals.

Each of the transducers 24, 26, 28 and each of the acceleration sensors 30, 32 have a signal processing unit 34 assigned to it that is provided to process a signal that is generated by the transducers 24, 26, 28 and acceleration sensors 30, 32, respectively, in response to the physiological parameter.

The signal processing units 34 of each of the acceleration sensors 30, 32 are provided to evaluate the acceleration signal regarding at least one out of frequency, amplitude, and signal pattern to derive an acceleration signal corresponding to at least one maternal physiological parameter, as will be explained in the following. A schematic functional diagram of the fetal monitoring system 10 is illustrated in FIG. 2. The two transducer units 16, 18 provide processed signals to a signal monitoring unit 36 via cabling or, alternatively, via a wireless connection.

The signal monitoring unit 36 of the fetal monitoring system 10 provides several options for displaying processed signals, as is commonly known. Further, the signal monitoring unit 36 comprises a signal selector unit 38 the function of which will be explained later on. The signal selector unit 38 is provided to carry out specific steps of a method for determining at least one maternal physiological parameter out of a heart rate and a breathing rate. To this end, the signal selector unit 38 comprises a software module 40 provided for carrying out these specific steps, wherein the steps are converted into a program code that is implemented in and executed by the signal selector unit 38 in the operational state.

Moreover, the fetal monitoring system 10 comprises a cross-channel verification unit 42 that is provided for determining a cross-channel verification between traces of two of the signals continuously over time. One of the two signals is the maternal heart rate signal MHR1, MHR2, MHR3 derived from any one of the acceleration sensors 30, 32 and the optical transducer 26. The other one of the two signals is the fetal heart rate FHR taken up by the transducer 28 designed as an ultrasonic Doppler sensor. In general, more than one ultrasonic Doppler sensor transducer could be employed for fetal monitoring, for instance in case of a multiple birth. Then, another cross-channel verification between the maternal heart rate signal MHR1, MHR2, MHR and a second fetal heart rate taken up by an additional ultrasonic Doppler sensor transducer would also be determined continuously over time.

Once the fetal monitoring system 10 is arranged as shown in FIG. 1 and put to operation, the transducers 24, 26, 28 and acceleration sensors 30, 32 provide their corresponding signals. The signal processing units 34 assigned to each one the acceleration sensors 30, 32 comprise means for evaluating the acceleration signals regarding at least one out of frequency, amplitude, and signal pattern to derive an acceleration signal corresponding to the at least one maternal physiological parameter, namely a maternal heart rate MHR2, MHR3.

The maternal heart rate MHR1, MHR2, MHR3 derived from any one of the acceleration sensors 30, 32 and the optical transducer 26 and the fetal heart rate FHR derived from the signal of the ultrasonic Doppler sensor are fed to the cross-channel verification unit 42 at a sample rate of the assigned signal processing units 34. The signal that is meant to represent the maternal heart rate MHR1, MHR2, MHR3 at the best is selected by the signal selector unit 38 based on multiple parameters like logical considerations, heart rate history, signal quality or other information. The cross-channel verification unit 42 compares a frequency of the selected maternal heart rate signal MHR1, MHR2, MHR3 and a fetal heart rate FHR signal from the ultrasonic Doppler sensor, and tracks a result over time. A pre-determined threshold for a difference in frequency is stored in a memory that the cross-channel verification unit 42 has access to. As long as the determined difference in frequency is larger than this pre-determined threshold, the signal selected by the signal selector unit 38 represents the correct maternal heart rate MHR1, MHR2, MHR3. Once the determined difference in frequency is smaller than the pre-determined threshold, the cross-channel verification unit 42 activates an alert signal 44 to indicate that the ultrasonic Doppler sensor signal might be affected by a maternal pulse activity. In response to the alert signal 44, a caregiver can adjust a position of the ultrasonic Doppler sensor at the maternal abdomen 14, so as the signal of the ultrasonic Doppler sensor to correctly correspond to the fetal heart rate FHR again. By that, a reliability and robustness of the fetal monitoring system 10 is significantly increased and the risk of intrapartum fetal mortality is reduced.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCE SYMBOL LIST

10 fetal monitoring system

12 mother

14 abdomen

16 transducer unit

18 transducer unit

20 housing

22 housing

24 transducer

26 transducer

28 transducer

30 acceleration sensor

32 acceleration sensor

34 signal processing unit

36 signal monitoring unit

38 signal selector unit

40 software module

42 cross-channel verification unit

44 alert signal

D display

MHR1 maternal heart rate signal

MHR2 maternal heart rate signal

MHR3 maternal heart rate signal

FHR fetal heart rate signal

1. A method for determining at least one maternal physiological parameter out of a heart rate and a breathing rate, comprising the following steps:
receiving a processed acceleration signal and at least one transducer signal corresponding to a fetal heart rate (FHR),
the at least one transducer signal being provided by at least one transducer provided for taking up physiological parameters and converting them into corresponding transducer signals, and
the processed acceleration signal being provided by a at least one signal processing unit provided to process said transducer signals and said acceleration signal and for evaluating the acceleration signal regarding at least one out of frequency, amplitude, and signal pattern to derive the processed acceleration signal corresponding to the at least one maternal physiological parameter,
the acceleration signal being acquired by at least one acceleration sensor provided for converting mechanical acceleration at the maternal abdomen into a corresponding acceleration signal, wherein the at least one acceleration sensor is different from the at least one transducer and wherein the at least one acceleration sensor in at least one operating mode, is rigidly mounted inside a housing that at least partially encompasses the at least one transducer;
determining and tracking a cross-channel verification between the processed acceleration signal and at least one transducer signal corresponding to a fetal heart rate (FHR); and
depending on a result of the cross-channel verification, activating an alert signal.

2. (canceled)

3. The method as claimed in claim 1, further comprising the step of:
selecting one out of the transducer signals and the processed accelerator signal, based on an evaluation of at least one out of signal history and signal quality.

4. (canceled)

5. The signal monitoring unit as claimed in claim 13, further comprising a signal selector unit for selecting one out of the transducer signals and the processed accelerator signal, based on an evaluation of at least one out of signal history and signal quality.

6. (canceled)

7. The fetal monitoring system as claimed in claim 14, wherein the at least one acceleration sensor is designed as a micro-electromechanical system.

8. The fetal monitoring system as claimed in claim 14, wherein the at least one acceleration sensor is provided for converting mechanical acceleration into corresponding acceleration signals in three substantially orthogonal directions.

9. A software module provided for carrying out the steps of the method of claim 3, wherein the steps are converted into a program code that is implementable in and executable by the signal monitoring unit.

10. (canceled)

11. The method as claimed in claim 1, further comprising the step of attaching at least two transducer units of a fetal monitoring system to a maternal abdomen, one of them comprising the at least one transducer, the other one of them comprising the at least one acceleration sensor, and each of them comprising a signal processing unit.

12. The method as claimed in claim 1, further comprising the step of evaluating the acceleration signal regarding at least one out of frequency, amplitude, and signal pattern to derive a processed acceleration signal corresponding to the at least one maternal physiological parameter.

13. A signal monitoring unit, comprising:
means for receiving a processed acceleration signal and at least one transducer signal corresponding to a fetal heart rate (FHR),
the at least one transducer signal being provided by at least one transducer provided for taking up physiological parameters and converting them into corresponding transducer signals, and
the processed acceleration signal being provided by at least one signal processing unit provided to process said transducer signals and said acceleration signal and for evaluating the acceleration signal regarding at least one out of frequency, amplitude, and signal pattern to derive the processed acceleration signal corresponding to the at least one maternal physiological parameter,
the acceleration signal being acquired by at least one acceleration sensor provided for converting mechanical acceleration at the maternal abdomen into a corresponding acceleration signal, wherein the at least one acceleration sensor is different from the at least one transducer, and wherein the at least one acceleration sensor, in at least one operating mode, is rigidly mounted inside a housing that at least partially encompasses the at least one transducer; and
a cross-channel verification unit for determining and tracking a cross-channel verification between the processed acceleration signal and at least one transducer signal corresponding to a fetal heart rate (FHR) and for activating an alert signal depending on a result of the cross-channel verification.

14. A fetal monitoring system comprising:
at least two transducer units, comprising:
at least one transducer provided for taking up physiological parameters and converting them into corresponding transducer signals,
at least one acceleration sensor provided for converting mechanical acceleration at the maternal abdomen into a corresponding acceleration signal, wherein the at least one acceleration sensor is different from the at least one transducer, and wherein the at least one acceleration sensor, in at least one operating mode, is rigidly mounted inside a housing that at least partially encompasses the at least one transducer, and
at least one signal processing unit provided to process said transducer signals and said acceleration signal and for evaluating the acceleration signal regarding at least one out of frequency, amplitude, and signal pattern to derive the processed acceleration signal corresponding to the at least one maternal physiological parameter; and
a signal monitoring unit as claim in claim 5 for processing the processed acceleration signal and at least one transducer signal.
